(19) United States
(12) Patent Application Publication
(10) Pub. No.: US 2026/0273433 A1
(43) Pub. Date: Sep. 17, 2026

(54) CONSTANT-TEMPERATURE DUAL-CYCLE MATERIAL SEPARATION METHOD AND USE THEREOF

(71) Applicant: Naizhong CUI, Datong (CN)

(72) Inventor: Naizhong CUI, Datong (CN)

(21) Appl. No.: 19/290,417

(22) Filed: Aug. 5, 2025

(30) Foreign Application Priority Data
Mar. 17, 2025 (CN) 202510316644.X

(51) Int. Cl. B01D 3/34; A61L 2/00; A61L 101/34; B01D 3/00; C12G 3/08

(52) U.S. Cl. CPC B01D 3/34; A61L 2/20; B01D 3/005; C12G 3/08; A61L 2101/34; A61L 2103/05

(57) ABSTRACT

Provided are a constant-temperature dual-cycle substance separation method and use thereof. In a fully-enclosed treatment system, a thermal control substance with a phase transition temperature between those of a low-phase-transition-temperature component and a high-phase-transition-temperature component in a substance to be treated is in contact with the low-temperature-treated substance for mass transfer and heat transfer, such that the low-phase-transition-temperature component in the low-temperature-treated substance is separated from the high-phase-transition-temperature component; and low-temperature dissipation of the treatment system is achieved through a substance cycle of the thermal control substance and a substance cycle of the low-temperature-treated substance.

### CROSS REFERENCE TO THE RELATED APPLICATIONS

This application is based upon and claims priority to Chinese Patent Application No. 202510316644.X, filed on Mar. 17, 2025, the entire contents of which are incorporated herein by reference.

### TECHNICAL FIELD

The present disclosure belongs to the technical field of substance separation, and in particular to a constant-temperature dual-cycle substance separation method and use thereof.

### BACKGROUND

Temperature control is crucial when substance separation and the other treatments are conducted based on different phase transition temperatures. Existing methods include directly or indirectly heating the substance in advance (such as chemical distillation), and then carrying out subsequent treatment such as substance separation. Usually, whole components of the low-temperature-treated substance need to be heated to a certain temperature prior to being used for subsequent treatment. In the actual treatment process, it is usually impossible to achieve uniform temperature rise of all components of the treated substance within a limited time and space, resulting in problems such as local low temperature and overheating. Meanwhile, the pretreatment process is time-consuming and energy-consuming, leading to a great increase in costs of equipment, management, and labor. Therefore, the existing methods have the disadvantages of low separation efficiency, high ineffective energy consumption, and uneven heating of the treated substance during preheating.

### SUMMARY

In view of this, the present disclosure aims at providing a constant-temperature dual-cycle substance separation method and use thereof. The method could create a “fully-enclosed, continuous, stable and controllable” microenvironment in a treatment system, allows that a substance to be treated could be uniformly heated, and results in high component separation efficiency, and low energy consumption.

To achieve the object above, the present disclosure provides the following technical solutions:

A constant-temperature dual-cycle substance separation method includes the following steps:
carrying out two cycles in a fully-closed treatment system, where the two cycles include a substance cycle of a high-temperature thermal control substance, and a substance cycle of a low-temperature-treated substance, the low-temperature-treated substance includes a low-phase-transition-temperature component and a high-phase-transition-temperature component, and the high-temperature thermal control substance has a phase transition temperature between a phase transition temperature of the low-phase-transition-temperature component and a phase transition temperature of the high-phase-transition-temperature component; where
the substance cycle of the low-temperature-treated substance is performed by: heating the high-temperature thermal control substance and then bring a heated high-temperature thermal control substance in contact with the low-temperature-treated substance for heat exchange, such that phase transition latent heat of the high-temperature thermal control substance during the heat exchange enables the low-phase-transition-temperature component in the low-temperature-treated substance to undergo first-order phase transition and then separation from the high-phase-transition-temperature component, and taking out the low-phase-transition-temperature component after the separation; and taking out the high-phase-transition-temperature component after the separation, or introducing the high-phase-transition-temperature component after the separation into a next cycle process as the low-temperature-treated substance; and
the substance cycle of the high-temperature thermal control substance is performed by: after heat exchange with the low-temperature-treated substance, reheating the high-temperature thermal control substance by reflux, and then introducing a reheated high-temperature thermal control substance into a next cycle process.

In some embodiments, each of the high-temperature thermal control substance, the low-phase-transition-temperature component, and the high-phase-transition-temperature component is a substance with a first-order phase transition property.

In some embodiments, the low-temperature-treated substance is fed in a feeding mode selected from the group consisting of batch feeding and unidirectional continuous cyclic feeding.

In some embodiments, the high-temperature thermal control substance is one selected from the group consisting of a gaseous substance and a liquid substance, and the gaseous substance is one selected from the group consisting of a gaseous azeotropic mixture and a gaseous non-azeotropic mixture.

In some embodiments, the low-temperature-treated substance exhibits fluidity;
once not exhibiting fluidity, the low-temperature-treated substance is pulverized to achieve the fluidity.

In some embodiments, under the condition that the substance with the first-order phase transition property in the fully-closed treatment system is gaseous, a phase transition temperature of the substance with the first-order phase transition property is changed by changing a pressure of the fully-closed treatment system.

In some embodiments, under the condition that a pressure of the fully-closed treatment system is a constant pressure, a temperature of the fully-closed treatment system is controlled by controlling a phase transition temperature property and/or a reflux speed of the high-temperature thermal control substance.

In some embodiments, under the condition that the high-temperature thermal control substance is the gaseous non-azeotropic mixture, a temperature of the fully-closed treatment system is controlled by controlling a proportion of each component in the high-temperature thermal control substance.

The present disclosure further provides use of the constant-temperature dual-cycle substance separation method as described in above technical solutions in substance separation, concentration, and surface treatment.

In some embodiments, the substance separation includes preparing Xanthoceras sorbifolium flower-flavoured brandy, the surface treatment includes microbial inactivation, and the microbial inactivation includes inactivating surface microorganisms of a wild and freshly-picked Datong daylily;
during the preparing the Xanthoceras sorbifolium flower-flavoured brandy, the high-temperature thermal control substance is a gaseous ethanol-water non-azeotropic mixture at a temperature of 80° C. to 85° C., and the low-temperature-treated substance is Xanthoceras sorbifolium flower dried at ambient temperature;
during the inactivating the surface microorganisms of the wild and freshly-picked Datong daylily, the high-temperature thermal control substance is the gaseous ethanol-water non-azeotropic mixture at a temperature of 80° C. to 85° C., and the low-temperature-treated substance is the wild and freshly-picked Datong daylily.

The present disclosure provides a constant-temperature dual-cycle substance separation method, including the following steps: carrying out two cycles in a fully-closed treatment system, where the two cycles include a substance cycle of a high-temperature thermal control substance, and a substance cycle of a low-temperature-treated substance, the low-temperature-treated substance includes a low-phase-transition-temperature component and a high-phase-transition-temperature component, and the high-temperature thermal control substance has a phase transition temperature between a phase transition temperature of the low-phase-transition-temperature component and a phase transition temperature of a high-phase-transition-temperature component; the substance cycle of the low-temperature-treated substance is performed by: heating the high-temperature thermal control substance and then bringing a heated high-temperature thermal control substance in contact with the low-temperature-treated substance for heat exchange, such that phase transition latent heat of the high-temperature thermal control substance during the heat exchange enables the low-phase-transition-temperature component in the low-temperature-treated substance to undergo first-order phase transition and separation from the high-phase-transition-temperature component, and taking out the low-phase-transition-temperature component after the separation, and taking out the high-phase-transition-temperature component after the separation, or entering the high-phase-transition-temperature component after the separation into a next cycle process as the low-temperature-treated substance; and the substance cycle of the high-temperature thermal control substance is performed by: after heat exchange with the low-temperature-treated substance, reheating the high-temperature thermal control substance by reflux, and then entering reheated high-temperature thermal control substance into a next cycle process.

In the present disclosure, the high-temperature thermal control substance with a phase transition temperature between the phase transition temperatures of the low-phase-transition-temperature component and the high-phase-transition-temperature component in the low-temperature-treated substance is in contact with a surface of the low-temperature-treated substance for mass transfer and heat transfer. A heat transfer direction is from a low-density high-temperature thermal control substance to a high-density low-temperature-treated substance, and a heat transfer mode is non-penetrating surface homogeneous mass transfer and heat transfer. In the process of mass transfer and heat transfer between the high-temperature thermal control substance and the low-temperature-treated substance, a direct contact surface therebetween is static without bubbles, overboiling and other phenomena, which is conducive to improving the efficiency of mass transfer and heat transfer, making the low-temperature-treated substance uniformly heated and thereby resulting in high separation efficiency of components. Moreover, only the surface of the low-temperature-treated substance is heated, and the temperature rise is limited by the phase transition temperature of the high-temperature thermal control substance, which has the advantages of high efficiency, low energy consumption, and accurate temperature control. Moreover, during dual cycle, the components could be separated more thoroughly and enriched, thereby improving the purity of each of collected components. The substance cycle of the high-temperature thermal control substance enables the high-temperature thermal control substance to exchange heat with the low-temperature-treated substance and take away low temperature to achieve low-temperature dissipation of the treatment system, thereby realizing a “fully-enclosed, continuous, stable and controllable” microenvironment of the treatment system, and the consumption of the high-temperature thermal control substance is reduced. In addition, due to the adoption of the fully-enclosed treatment, the zero emission of pollutants could be achieved.

### BRIEF DESCRIPTION OF THE DRAWINGS

FIG. 1 shows a schematic diagram of working principle of a constant-temperature dual-cycle substance separation method according to an embodiment of the present disclosure, with the following reference signs: 1—a treatment system; 2—a high-temperature thermal control substance generating device; 3—a high-temperature thermal control substance; 4—a low-temperature-treated substance, 5—a low-phase-transition-temperature component; 6—a high-phase-transition-temperature component; 7—a first external pipeline; 8—a first one-way drive device; 9—a second external pipeline; 10—a second one-way drive device; and 11—a multilayer horizontal groove; and

FIG. 2 shows a schematic diagram of a method for preparing Xanthoceras sorbifolium flower-flavoured brandy according to an embodiment of the present disclosure, with the following reference signs: 21—a distillation still, 22—a rectification tower, 23—a cooling tower, 24—a primary distillate, 25—a gaseous ethanol-water non-azeotropic mixture; 26—a tower plate, 27—Xanthoceras sorbifolium flower dried at ambient temperature; 28—a low-boiling-point fraction collecting device, 29—a high-boiling-point fraction collecting device; 210—a liquor collecting device; and 211—a valve.

### DETAILED DESCRIPTION OF THE EMBODIMENTS

### Definition

Unless otherwise specified, in the disclosure, the term “high” in “high-temperature thermal control substance” refers to “a higher temperature” than that of “low-temperature-treated substance”; conversely, the term “low” in “low-temperature-treated substance” refers to “a lower temperature” than that of “high-temperature thermal control substance”.

Unless otherwise specified, in the disclosure, the term “low-phase-transition-temperature” in “low-phase-transition-temperature component” refers to “a lower phase transition temperature” than that of “high-phase-transition-temperature component”; conversely, the term “high-phase-transition-temperature” in “high-phase-transition-temperature component” refers to “a higher phase transition temperature” than that of “low-phase-transition-temperature component”.

To avoid any clarity issues, the term “high-temperature thermal control substance” is also referred to as “thermal control substance” in claims; and the term “low-temperature-treated substance” is also referred to as “substance to be treated” in claims. To avoid any clarity issues, the term “low-phase-transition-temperature component” is also referred to as “first-phase-transition-temperature component” in claims; and the term “high-phase-transition-temperature component” is also referred to as “second-phase-transition-temperature component” in claims.

The present disclosure provides a constant-temperature dual-cycle substance separation method, including the following steps:
carrying out two cycles in a fully-closed treatment system, where the two cycles include a substance cycle of a high-temperature thermal control substance, and a substance cycle of a low-temperature-treated substance, the low-temperature-treated substance includes a low-phase-transition-temperature component and a high-phase-transition-temperature component, and the high-temperature thermal control substance has a phase transition temperature between a phase transition temperature of the low-phase-transition-temperature component and a phase transition temperature of the high-phase-transition-temperature component;
the substance cycle of the low-temperature-treated substance is performed by: heating the high-temperature thermal control substance and then bringing a heated high-temperature thermal control substance in contact with the low-temperature-treated substance for heat exchange, such that phase transition latent heat of the high-temperature thermal control substance during the heat exchange enables the low-phase-transition-temperature component in the low-temperature-treated substance to undergo first-order phase transition and separation from the high-phase-transition-temperature component, and taking out the low-phase-transition-temperature component after the separation, and taking out the high-phase-transition-temperature component after the separation, or entering the high-phase-transition-temperature component after the separation into a next cycle process as the low-temperature-treated substance; and
the substance cycle of the high-temperature thermal control substance is performed by: after heat exchange with the low-temperature-treated substance, reheating the high-temperature thermal control substance by reflux, and then entering reheated high-temperature thermal control substance into a next cycle process.

In the present disclosure, unless otherwise specified, there is no special requirement on the source of the raw materials used, and commercial products familiar to those skilled in the art may be used.

In an embodiment, each of the high-temperature thermal control substance, the low-phase-transition-temperature component, and the high-phase-transition-temperature component is a substance with a first-order phase transition property. The phase transition of the substance with the first-order phase transition property in the treatment system occurs at a constant temperature, which depends on an internal environment pressure of the treatment system. The present disclosure aims at controlling an upper temperature limit, i.e., controlling a temperature of the treatment system below the phase transition temperature of the high-temperature thermal control substance. For example, under the condition that a concentration of ethanol is 95.6% and a concentration of water is 4.4%, an azeotropic point of an azeotropic mixture of gaseous ethanol and water (i.e., the phase transition temperature of the azeotropic mixture) is 78.13° C., and thus the temperature of a cycling treatment system is controlled below 78.13° C. As the pressure is constant, the proportion of the azeotrope component is unchanged and cycling treatment is adopted, although the temperature of the treatment system changes with the above factors, the temperature of the treatment system cannot increase to be above the phase transition temperature of high-temperature thermal control substance.

In an embodiment, the high-temperature thermal control substance is a gaseous substance or liquid substance. In an embodiment, the gaseous substance or liquid substance is a pure substance or a mixture. In an embodiment, the gaseous substance is a gaseous azeotropic mixture or a gaseous non-azeotropic mixture, which is the gaseous non-azeotropic mixture in a specific embodiment. The gaseous non-azeotropic mixture, whose boiling range could be greatly changed by adjusting the components, is thus used as the high-temperature thermal control substance to provide a simple and easy temperature control means.

In an embodiment, under the condition that the high-temperature thermal control substance is the gaseous substance, the high-temperature thermal control substance may be a high-temperature steam. Under the condition that the high-temperature thermal control substance is the liquid substance, the high-temperature thermal control substance may be a high-temperature liquid metal. During cycling, the high-temperature thermal control substance may undergo instantaneous phase transition locally, but due to the cycling treatment, the high-temperature thermal control substance generally only releases part of phase transition latent heat, and usually do not undergo phase transition. For example, high-temperature liquid copper is used as a high-temperature thermal control substance to separate the low-temperature-treated substance containing aluminum and iron. The aluminum is melted first, mixed with copper and thus is separated from iron, and then cooling is carried out to make the copper undergo phase transition and solidification to be separated from the aluminum, thus removing aluminum. Afterwards, the cooper is added to the next cycle process as a high temperature thermal control substance.

In the present disclosure, the high-temperature thermal control substance (also referred to as “thermal control substance”) when contacting with the low-temperature-treated substance, acts a high-temperature heat source, and the low-temperature-treated substance (also referred to as “substance to be treated”) acts as a low-temperature source. Once there is a surface contact between the high-temperature thermal control substance and the low-temperature-treated substance, mass and heat transfer occur and “low-temperature dissipation” is realized.

In an embodiment, the low-phase-transition-temperature component in the low-temperature-treated substance is gaseous, liquid-state, or solid-state, and the high-phase-transition-temperature component is liquid-state, or solid-state. In the process of cyclic separation, the low-phase-transition-temperature component may be separated by solid-liquid phase transition and liquid-gas phase transition, and the high-phase-transition-temperature component may be separated by solid-liquid phase transition.

The main difference between the first-order phase transition and the second-order phase transition lies in the continuity of a thermodynamic function and its derivative in the phase transition process and whether it is accompanied by the change(s) of latent heat and volume. The present disclosure is only applicable to a substance that is able to undergo first-order phase transition, accompanied by the release or absorption of the phase transition latent heat. With the partial release of the phase transition latent heat of the high-temperature thermal control substance, only the low-phase-transition-temperature component in the low-temperature-treated substance is released. As the treatment process involves cycling, flow, contact, mass transfer, and heat transfer, the high-phase-transition-temperature component in the low-temperature-treated substance could not undergo the same phase transition as the low-phase-transition-temperature component due to the existence of the phase transition latent heat, and thus is separated in liquid or solid form.

In the substance cycle process of the high-temperature thermal control substance, the high-temperature thermal control substance exchanges heat with the low-temperature-treated substance and takes away the low temperature, which achieves the ‘low temperature dissipation’ of the treatment system, thereby realizing a “fully-enclosed, continuous, stable and controllable” microenvironment of the treatment system.

In an embodiment, the low-temperature-treated substance is fed in a feeding mode of batch feeding or unidirectional continuous cyclic feeding. In a specific embodiment, the feeding mode is batch feeding. The above feeding modes could increase direct contact area between the high-temperature thermal control substance and the low-temperature-treated substance, or improve the surface contact efficiency in the cyclic flowing process. In the cycling process the low-temperature-treated substance, the high-temperature thermal control substance contacts different surfaces of the low-temperature-treated substance. For example, under the condition that the low-temperature-treated substance is in a liquid state, the flowing process is equivalent to constantly changing interface molecules in contact with the high-temperature thermal control substance, so it is equivalent to increasing the surface area of the liquid, thereby greatly improving the heat exchange efficiency.

In an embodiment, under the condition that the low-temperature-treated substance is fed by unidirectional continuous cyclic feeding, the incompletely treated low-temperature-treated substance is introduced into the treatment system again as a raw material to enter the next cycling process at the end of each cycling process. Therefore, in the cyclic separation process, there is no need to subject the low-temperature-treated substance to complete separation in each cyclic separation process.

In an embodiment, under the condition that the high-temperature thermal control substance is a gaseous azeotropic mixture, an azeotropic point of the gaseous azeotropic mixture is between phase transition temperatures of the low-phase-transition-temperature component and the high-phase-transition-temperature component in the low-temperature-treated substance. The gaseous azeotropic mixture has the advantages of economy, easy availability, fast mass and heat transfer speeds, and high efficiency. Under the condition that the high-temperature thermal control substance is a gaseous non-azeotropic mixture, its difference than the gaseous azeotropic mixture is that the gaseous non-azeotropic mixture has a dynamic boiling range, and a wider temperature control range could be obtained by adjusting the proportion of each component in the gaseous non-azeotropic mixture at a constant pressure. In the present disclosure, the component of the gaseous azeotropic mixture could be selected at will, and the selection is made according to principle that the component selected for the gaseous azeotropic mixture has the characteristics of the high-temperature thermal control substance, i.e., the substance with a first-order phase transition property, and the phase transition temperature thereof between the phase transition temperatures of the low-phase-transition-temperature component and the high-phase-transition-temperature component in the low-temperature-treated substance, which may be a multi-component mixture.

In an embodiment, a heat conductivity coefficient of the high-temperature thermal control substance is determined according to the selected high-temperature thermal control substance, and in a specific embodiment, the heat conductivity coefficient is determined according to a ratio of ethanol to water in the selected azeotropic mixture of gaseous ethanol and water. The heat conductivity coefficient of the high-temperature thermal control substance is high, which is conducive to improving the response speed, efficiency, and long-term stability of temperature control by the high-temperature thermal control substance.

In an embodiment, timely monitoring and replacement of the high-temperature thermal control substance are configured to prevent the performance attenuation of the high-temperature thermal control substance, such as phase transition enthalpy reduction and phase transition temperature shift, from affecting the temperature control effect. In the present disclosure, the heat conductivity coefficient could be measured in real time by sampling and measuring or by using a heat conductivity coefficient tester, with the principle that the heat conductivity coefficient is generally measured by a transient heat source method under non-steady-state methods. There are many factors affecting the heat conductivity coefficient, for example, the factors causing the change of the heat conductivity coefficient include temperature, pressure, purity of water, the state of water, etc., and thus the high-temperature thermal substance could be directly replaced after the test shows inappropriate condition.

In an embodiment, the low-temperature-treated substance exhibits fluidity. Under the condition that the low-temperature-treated substance does not exhibit fluidity, the low-temperature-treated substance is pulverized to achieve the fluidity. Through the pretreatment means of pulverizing, the low-temperature-treated substance without fluidity is transformed into the substance with fluidity, thus increasing the surface area thereof.

In an embodiment, under the condition that the substance with the first-order phase transition property in the treatment system is gaseous, the phase transition temperature of the substance with the first-order phase transition property is changed by changing a pressure of the treatment system, thus controlling the temperature of the treatment system, with the advantages of simplicity, convenience, and quickness.

In another embodiment, the azeotropic point of the gaseous azeotropic mixture serving as the high-temperature thermal control substance is changed by changing the pressure of the treatment system, thus accurately controlling an upper temperature limit of the treatment system.

In an embodiment, the phase transition temperature of the high-temperature thermal control substance is adjusted by vacuum distillation or pressurizing the treatment system, and the phase transition temperature of the gaseous high-temperature thermal control substance is reduced by vacuum distillation, and the phase transition temperature of the high-temperature thermal control substance is improved by pressurizing the treatment system, thereby achieving temperature control through pressure change.

In the present disclosure, a quantitative relationship between the pressure and temperature control changes with the selection of the high-temperature thermal control substance. For example, the temperature of the gaseous ethanol-water non-azeotropic mixture changes with a ratio of ethanol to water at atmospheric pressure. A lower proportion of the ethanol component results in a higher temperature of the gaseous ethanol-water non-azeotropic mixture, which varies from 78.13° C. to 100° C. The boiling point of ethanol is 78.3° C., while the boiling point of water is 100° C., and the azeotropic point of ethanol-water azeotropic mixture is 78.13° C., where a concentration of ethanol is 95.6%, and a concentration of water is 4.4%. The azeotropic point (boiling range) of the non-azeotropic mixture changes with the change of component proportion.

In an embodiment, heating the high-temperature thermal control substance refers to generating high-temperature thermal control substance in a high-temperature thermal control substance generating device. In an embodiment, the high-temperature thermal control substance generating device is a distillation still or a melting furnace. In an embodiment, the treatment system is a rectification tower or a melting furnace, and in a specific embodiment, it is the rectification tower. In an embodiment, the high-temperature thermal control substance generating device communicates with the treatment system. In an embodiment, the treatment system is provided with a collecting device of a low-phase-transition-temperature component in the treated substance. In an embodiment, the treatment system is provided with a collecting device of a high-phase-transition-temperature component in the treated substance. In an embodiment, the treatment system is provided with two sets of external pipelines, and a one-way drive device, one set of the external pipelines is used for the substance cycle of the low-temperature-treated substance, and the other set of the external pipelines is used for the cycle of the high-temperature thermal control substance. In an embodiment, the one-way drive device is an air pump or an air suction fan, and in a specific embodiment, the one-way drive device is the air pump. According to the present disclosure, a high-temperature thermal control substance forms unidirectional cycling power through the one-way drive device. In the present disclosure, the internal and external pipelines in the treatment system form a cycle under the action of one-way drive to achieve the temperature control of the treatment system. If the cyclic flow of the high-temperature thermal control substance is only formed in the treatment system, the property of the high-temperature thermal control substance in the treatment process, including the real-time consumption of the phase transition latent heat of the high-temperature thermal control substance, could not be controlled. The external pipelines aim at assisting control and building a fully-enclosed cycle, thus achieving zero emission of pollutants.

In an embodiment, when the high-temperature thermal control substance after heating the low-temperature-treated substance is collected, a valve is arranged between the external pipelines and the high-temperature thermal control substance generating device, and a cooling device and a collecting device are arranged between the external pipelines. In an embodiment, the cooling device is a cooling tower.

In an embodiment, under the condition that the pressure of the treatment system is a constant pressure, it is simple and fast to control the temperature of the treatment system by controlling the properties and/or reflux speed of the high-temperature thermal control substance. Upper temperature limits of all substances in the treatment system are controlled by the high-temperature thermal control substance to be not higher than the phase transition temperature of the high-temperature thermal control substance.

In the present disclosure, the constant pressure refers to the pressure selected by the treatment system, for example, the atmospheric distillation is selected in the rectification tower, and vacuum distillation under different pressure levels may also be selected. The constant pressure of the treatment system could be effectively controlled by external equipment, such as vacuum distillation equipment.

In the present disclosure, the temperature of the gaseous high-temperature thermal control substance formed in the treatment system changes with different constant pressures, which is directly related to the phase transition temperatures of the high-temperature thermal control substance and the low-temperature-treated substance in the treatment system. For example, if the high-temperature thermal control substance is water, when the pressure of the treatment system is 101.3 kPa, the boiling point of the water is 100° C.; when the pressure of the treatment system drops to 12.34 kPa, the boiling point of the water is 50° C.; and when the pressure of the treatment system is 198.5 kPa, the boiling point of the water is 121.3° C.

In an embodiment, under the condition that the high-temperature thermal control substance is a gaseous non-azeotropic mixture, the temperature of the treatment system is controlled by controlling the proportion of each component in the high-temperature thermal control substance. The temperature of the treatment system is changed by controlling the proportion of each component in the gaseous non-azeotropic mixture of the high-temperature thermal control substance.

In the present disclosure, according to the phase transition temperature property of the high-temperature thermal control substance, for example, under the condition that the high-temperature thermal control substance is a gaseous mixture, the boiling range of the mixture could be changed by changing the proportion of each component in the mixture. Except for the azeotropic mixture, the mixture has no boiling point, but boiling range called herein.

In the present disclosure, the composition of the gaseous non-azeotropic mixture is related to raw material selection in the gaseous non-azeotropic mixture generating device, and the type and proportion of each component determine the boiling range of the gaseous non-azeotropic mixture. Raw materials to generate the gaseous non-azeotropic mixture are usually selected within a range. For example, as long as both ethanol and water exist, the gaseous ethanol-water non-azeotropic mixture could be formed, and the boiling range of the formed gaseous ethanol-water non-azeotropic mixture is below 100° C. In examples of the present disclosure, the primary distillate of fruit fermentation broth is selected as a raw material for generating the gaseous ethanol-water non-azeotropic mixture, which contains many impurities; however, when a volume proportion of the ethanol ranges from 26% to 29%, the gaseous ethanol-water non-azeotropic mixture could be effectively formed, and the cycling temperature could be stably controlled at about 80° C. When the proportion of ethanol is about 2%, the temperature of the formed gaseous non-azeotropic mixture is increased to above 95° C. due to the low proportion of ethanol.

In an embodiment, the temperature of the treatment system is controlled to be relatively constant by controlling the proportion of each component in the gaseous non-azeotropic mixture in the cycling separation process.

By adjusting treatment system pressure and changing the properties of the high-temperature thermal control substance, the temperature could be changed or maintained constant, with the advantages of accuracy, simplicity, convenience, and rapidness. The pressure of the treatment system could be adjusted by external equipment of the treatment system, for example, the vacuum distillation may be achieved by means of an air pump. The degree of pressure control depends on the requirements of substance separation and related treatment in the treatment system. For example, high pressure must not be used for the treatment of Xanthoceras sorbifolium flowers, otherwise, the food properties of Xanthoceras sorbifolium flowers are changed (Xanthoceras sorbifolium flowers become ‘mud’ under high pressure). By changing the properties of the high-temperature thermal control substance, the temperature could be controlled to be changed or kept constant. For example, in Example 1 of the present disclosure, when the proportion of ethanol in cyclic reflux stream decreases, the proportion of ethanol in the high-temperature thermal control substance generating device decreases, thereby decreasing the proportion of ethanol in the generated gaseous ethanol-water non-azeotropic mixture. Meanwhile, the temperature of the treatment system increases, the upper limit of which is the boiling point temperature of water, i.e., 100° C. Correspondingly, the temperature of the treatment system could be kept constant by controlling the real-time reflux of the ethanol to the high-temperature thermal control substance generating device and keeping the ethanol within a certain concentration range in the high-temperature thermal control substance generating device.

According to the present disclosure, the gaseous non-azeotropic mixture is used as the high-temperature thermal control substance, which has the advantage of high penetrability while ensuring accurate temperature control, and the penetrability of the gaseous substance is higher than that of the liquid substance. For example, when the liquid water is in surface contact with an object, it is often unable to effectively contact with the surface of the object due to tiny bubbles formed on the surface of the object, which greatly affects killing surface microorganisms of the object, resulting in that the microorganisms survive due to the protection of tiny bubbles. Once using a gaseous substance, no protective layer similar to the tiny bubbles is formed, and thus microorganisms could be effectively and thoroughly killed. The use of a gaseous non-azeotropic mixture as the high-temperature thermal control substance could meet the requirements of high penetrability and accurate temperature control during the temperature-controlled microorganisms inactivation in food processing, which is beneficial to the surface contact, cycling, mass transfer and heat transfer between the high-temperature thermal control substance and the low-temperature-treated substance.

In an embodiment, the low-temperature-treated substance is placed in a multilayer horizontal groove for cycling. The multilayer horizontal groove could increase the contact area between the low-temperature-treated substance and the high-temperature thermal control substance, and improve the mass transfer and heat transfer efficiency in the process of cyclic flowing. The heat exchange between a lower surface and a side surface of the multilayer horizontal groove and the high-temperature thermal control substance plays a role in preheating the low-temperature-treated substance that flows in the multilayer horizontal groove in one direction. In the present disclosure, the multilayer horizontal groove here is only to provide a possibility to optimize the effective treatment when the treatment capacity of the low-temperature-treated substance increases. The qualitative and quantitative indexes such as the number of layers, the size and the shape of the multilayer horizontal groove need to be determined according to the property of the low-temperature-treated substance, the treatment capacity, and a design target of a treatment device, and there is no special restrictions on this in the present disclosure.

In an embodiment, after the low-phase-transition-temperature component in the low-temperature-treated substance is separated from the high-phase-transition-temperature component, a small amount of known high-temperature thermal control substance impurities could be removed by changing pressure or temperature, which has the advantages of simplicity and convenience. As the phase transition temperature of the high-temperature thermal control substance is different from those of the low-phase-transition-temperature component and the high-phase-transition-temperature component in the low temperature-treated substance, when a small amount of the known high-temperature thermal control substance is mixed with the low-phase-transition-temperature component or the high-phase-transition-temperature component in the low temperature-treated substance, by only effectively controlling the pressure or temperature change of an external circulation pipeline, the phase transition temperature or temperature of the high-temperature thermal control substance could be changed, thereby being separated. If the purity requirement is high, the existing purification technology may be adopted in the design of the external circulation pipelines.

In an embodiment, under the condition that the low-temperature-treated substances involves multi-component separation, high-temperature thermal control substances with different phase transition temperatures could be used for step-by-step separation, the step-by-step separation is performed by only replacing high-temperature thermal control substances, in no need of constructing a multi-tray fractionating tower, thus reducing the production cost and improving the production security. The constant-temperature dual-cycle substance separation method provided by the present disclosure has low requirements on equipment precision, and could greatly reduce the production cost. The replacement of the high-temperature thermal control substance depends on the low-temperature-treated substance. For example, when the temperatures at which two groups of different low-phase-transition-temperature components and high-phase-transition-temperature components in the low-temperature-treated substance are separated need to be controlled at 80° C. and 100° C., the control could be achieved by replacing high-temperature thermal control substances with corresponding phase transition temperature, for example, replacing the ethanol-water non-azeotropic mixture with pure water.

In an embodiment, the high-temperature thermal control substance, the low-temperature-treated substance, and the separated low-phase-transition-temperature component and high-phase-transition-temperature component are all subjected to fully-enclosed cycling treatment, thereby achieving zero emission of pollutants.

In an embodiment, a device for fully-enclosed cycling treatment is a fully-enclosed fluid pollutant purification device disclosed in CN115569474A (incorporated by reference).

In the present disclosure, the high-temperature thermal control substance and the low-temperature-treated substance are in surface contact for mutual mass transfer and heat transfer during their respective unidirectional cycling. A heat transfer direction is from a low-density high-temperature thermal control substance to a high-density low-temperature-treated substance. In some embodiments, the heat transfer mode is non-penetrating surface homogeneous mass transfer and heat transfer, the direct contact surface between the high-temperature thermal control substance and the low-temperature-treated substance is static during the mass transfer and heat transfer, without generating bubbles, over boiling and other phenomena, which is conducive to improving the mass transfer and heat transfer efficiency.

Because the phase transition temperature of the substance with the first-order phase transition property in the treatment system is constant, the heat absorbed or released when the substance transforms from one phase to another under the same temperature and pressure, i.e., the phase transition latent heat, is related to the type of the substance, a phase transition type, and energy conversion in the phase transition process, and only the surface of the low-temperature-treated substance is heated in the treatment process, and the temperature rise is limited by the phase transition temperature of the high-temperature thermal control substance. Therefore, the constant-temperature dual-cycle substance separation method provided by the present disclosure has the advantages of high efficiency, low energy consumption, accurate temperature control and the like, which makes the energy consumption in the treatment process reduced greatly.

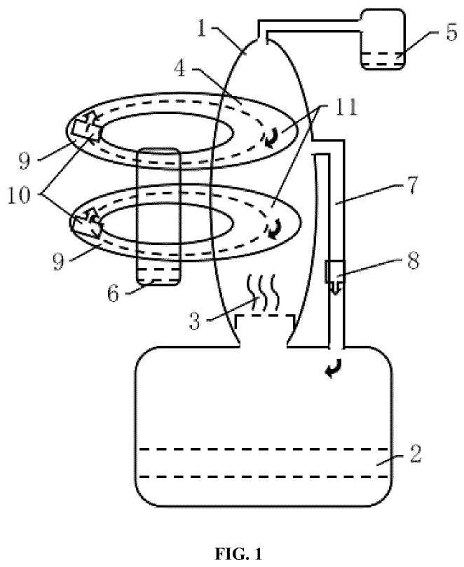

FIG. 1 shows a schematic diagram of working principle of the constant-temperature dual-cycle substance separation method according to an embodiment of the present disclosure. As shown in FIG. 1, in the treatment system 1, a phase transition temperature of a high-temperature thermal control substance 3 generated by heating in a high-temperature thermal control substance generating device 2 is between those of a low-phase-transition-temperature component 5 and a high-phase-transition-temperature component 6 in a low-temperature-treated substance 4. During treatment, after the generation of the high-temperature thermal control substance 3, a substance cycle of the high-temperature thermal control substance 3 between the treatment system 1 and a first external pipeline 7 is formed under the action of a first one-way drive device 8 in the first external pipeline 7. After the low-temperature-treated substance 4 is added, under the action of a second one-way drive device 10 in a second external pipeline 9, the low-temperature-treated substance 4 flows on a multilayer horizontal groove 11, thereby forming a substance cycle of the low-temperature-treated substance 4 between the treatment system 1 and the second external pipeline 9. The substance cycle of the high-temperature thermal control substance 3 and the substance cycle of the low-temperature-treated substance 4 are circulated in the treatment system 1, during which, the surface contact, cycling, mass transfer and heat transfer between the high-temperature thermal control substance 3 and the low-temperature-treated substance 4 are conducted, such that the low-phase-transition-temperature component 5 in the low-temperature-treated substance 4 is separated from the high-phase-transition-temperature component 6 due to the phase transition temperature limitation, thereby removing a small amount of the known high-temperature thermal control substance(s) that may be mixed in the low-phase-transition-temperature component 5 and the high-phase-transition-temperature component 6, and finally completing the processes such as separation, enrichment of components in the low-temperature-treated substance, or food processing. During this process, the temperature of the treatment system 1 could be controlled by controlling pressure change of the treatment system 1 or property change of the high-temperature thermal control substance 3. The low-phase-transition-temperature component 5, due to its low phase transition temperature, is firstly separated and collected in the treatment process. The high phase transition temperature 6 could be separated and taken out at any time in the second external pipeline 9 or enters the next substance cycle of the low-temperature-treated substance 4 as the low-temperature-treated substance 4. During treatment, due to the limitation of the phase transition temperature, the highest temperature in the treatment system 1 is controlled below the phase transition temperature of the high-temperature thermal control substance 3. The method provided by the present disclosure has the advantages of high efficiency, low energy consumption, and accurate temperature control, and meanwhile, the zero emission of pollutants is achieved by adopting fully-enclosed cycling.

According to the present disclosure, the substance separation is carried out through constant-temperature dual-cycle treatment, and the phase transition temperature of the substance with the first-order phase transition property is constant in a system with the same internal environmental pressure, and is related to an internal environmental pressure. According to the constant-temperature dual-cycle substance separation method provided by the present disclosure, in a case that the internal environment pressure is constant, a substance with a phase transition temperature between those of the low-phase-transition-temperature component and the high-phase-transition-temperature component in the low-temperature-treated substance is used as the high-temperature thermal control substance, and after heating, the surface contact, cycle, mass transfer, and heat transfer are carried out by controlling the dual cycle in the same treatment system, i.e., the substance cycle of the high-temperature thermal control substance and the substance cycle of the low-temperature-treated substance, such that the low phase transition component in the low-temperature-treated substance is separated from the high phase transition component due to phase transition temperature limitation. Moreover, after separation, a small amount of the known high-temperature thermal control substance(s) in components could be removed for further purification. The substance cycle of the low-temperature-treated substance is implemented in a manner of batch feeding or unidirectional continuous cyclic feeding, to increase the direct contact area between the high-temperature thermal control substance and the low-temperature-treated substance, or improving the surface contact efficiency in the cycling process. Under the condition that the unidirectional continuous cyclic feeding is adopted, at the end of each cycle separation, the low-temperature-treated substance that is not completely treated may serve as a raw material and is fed into the treatment system again with the cycle separation process, thereby entering the next cycle separation. Therefore, there is no need to subject the low-temperature-treated substance to thorough separation for each cycle separation. Transient extraction with only the gaseous ethanol-water non-azeotropic mixture could not concentrate an aromatic substance, and meanwhile, a large amount of gaseous ethanol-water non-azeotropic mixture needs to be consumed, resulting in failure to achieve a high level of restoration of processed food. If the ethanol-water non-azeotropic mixture alone is used for extraction, there is a risk of introducing allergic substances. According to the constant-temperature and dual-cycle substance separation method provided by the present disclosure, the components could be separated more thoroughly and enriched through multiple cycle processes of the substance cycle of the high-temperature thermal control substance and the substance cycle of the low-temperature-treated substance, the purity of the collected component products is improved, the “fully-enclosed, continuous, stable and controllable” microenvironment of the treatment system could be achieved, and the consumption of the high-temperature thermal control substance is reduced.

The ethanol-water non-azeotropic mixture shows broad-spectrum microbial inactivation effects, and has the penetrability of ethanol and high-temperature water steam, microbial protein denaturation capability, and enhanced microbial inactivation through latent heat release. Compared with single use of high-temperature water steam for microbial inactivation, the temperature is greatly reduced, and compared with single use of ethanol solutions with different concentrations for microbial inactivation, the irreversible microbial inactivation by releasing latent heat is added, and the problem that the microorganisms could not be completely inactivated due to the reversibility of protein denaturation caused by ethanol is solved. Moreover, the constant-temperature dual-cycle substance separation method could provide a “fully-enclosed, continuous, stable and controllable” microenvironment, which could not be achieved by using the ethanol-water non-azeotropic mixture alone. For example, when the gaseous ethanol-water non-azeotropic mixture is in contact with the low-temperature-treated substance, only “conduction from high temperature to low temperature” could be achieved, but low-temperature dissipation could not be achieved. If the low temperature of the microenvironment could not dissipate or dissipates too slowly, with the continuous addition of the low-temperature-treated substance, the temperature of the microenvironment would gradually decrease, and the inactivation effect thus decreases gradually, resulting in that some microorganisms survive. Meanwhile, the high-temperature thermal control substance may also undergo phase transition in a larger amount, making it impossible to acquire a continuous and stable microenvironment; and microenvironmental imbalance would lead to the failure of thorough microbial inactivation. According to the constant-temperature dual-cycle substance separation method provided by the present disclosure, through the substance cycle of the high-temperature thermal control substance, the high-temperature thermal control substance could take away the low temperature when exchanging heat with the low-temperature-treated substance, thus achieving the low-temperature dissipation of the treatment system, and realizing a “fully-enclosed, continuous, stable and controllable” microenvironment in the treatment system to thoroughly inactivate the microorganisms. Therefore, it has significant advantages to inactivate microorganisms by the constant-temperature dual-cycle substance separation method based on the gaseous ethanol-water non-azeotropic mixture.

The present disclosure further provides use of the constant-temperature dual-cycle substance separation method as described in above technical solutions in substance separation, concentration, and surface treatment.

In an embodiment, the substance separation includes preparing Xanthoceras sorbifolium flower-flavoured brandy. In an embodiment, the surface treatment includes microbial inactivation. In an embodiment, the microbial inactivation includes inactivating surface microorganisms of wild and freshly-picked Datong daylily.

In an embodiment, in the process of preparing the Xanthoceras sorbifolium flower-flavoured brandy, the high-temperature thermal control substance is a gaseous ethanol-water non-azeotropic mixture, the low-temperature-treated substance is Xanthoceras sorbifolium flower dried at ambient temperature, and a temperature of the gaseous ethanol-water non-azeotropic mixture ranges from 80° C. to 85° C. In a specific embodiment, the temperature of the gaseous ethanol-water non-azeotropic mixture is 85° C.

In an embodiment, in the process of inactivating surface microorganisms of wild and freshly-picked Datong daylily, the high-temperature thermal control substance is a gaseous ethanol-water non-azeotropic mixture, the low-temperature-treated substance is wild and freshly-picked Datong daylily, and the temperature of the gaseous ethanol-water non-azeotropic mixture ranges from 80° C. to 85° C. In a specific embodiment, the temperature of the gaseous ethanol-water non-azeotropic mixture is 85° C.

The following clearly and completely describes the technical solutions in the present disclosure in conjunction with the examples in the present disclosure, but the technical solutions should not be construed as limiting the scope of the present disclosure.

### Example 1 Preparation of Xanthoceras sorbifolium Flower-Flavoured Brandy

In this example, based on a constant-temperature dual-cycle substance separation method, primary distillate obtained by distilling grape fermentation broth was used to treat Xanthoceras sorbifolium flower dried at ambient temperature to obtain Xanthoceras sorbifolium flower-flavoured brandy with a unique flavor. In the treatment process, harmful low-phase-transition-temperature components such as methanol and acetaldehyde and high-phase-transition-temperature components such as higher alcohol that causes sensory discomfort were removed from the primary distillate and the Xanthoceras sorbifolium flower dried at ambient temperature, the original aromatic components such as alcohol, acid and ester in volatile components of the primary distillate and volatile components of the Xanthoceras sorbifolium flower dried at ambient temperature were introduced into the gaseous ethanol-water non-azeotropic mixture. In the cycling treatment process, as a main substance was the gaseous ethanol-water non-azeotropic mixture, the introduction of a small amount of impurities had little influence on the boiling range of the gaseous ethanol-water non-azeotropic mixture. In a stable state, on the one hand, the gaseous ethanol-water non-azeotropic mixture was used as a high-temperature thermal control substance to control the temperature of the treatment system to be constant and not higher than the boiling range of the gaseous ethanol-water non-azeotropic mixture in the cycling process of the treatment system and the external pipeline; on the other hand, the original aromatic components of the Xanthoceras sorbifolium flower dried at ambient temperature were introduced into the gaseous ethanol-water non-azeotropic mixture, and then cooled and collected to form the Xanthoceras sorbifolium flower-flavoured brandy with a unique flavor. In addition, the existing data show that the boiling range of the gaseous ethanol-water non-azeotropic mixture is affected by the proportion of ethanol to water. When the mass percentage of ethanol in the gaseous ethanol-water non-azeotropic mixture is higher than 82.8%, the temperature of the system can be controlled below 80° C., and compared with steam treatment at 100° C. under the same environmental pressure, the treatment temperature is greatly reduced, and meanwhile accurate temperature control could also be achieved.

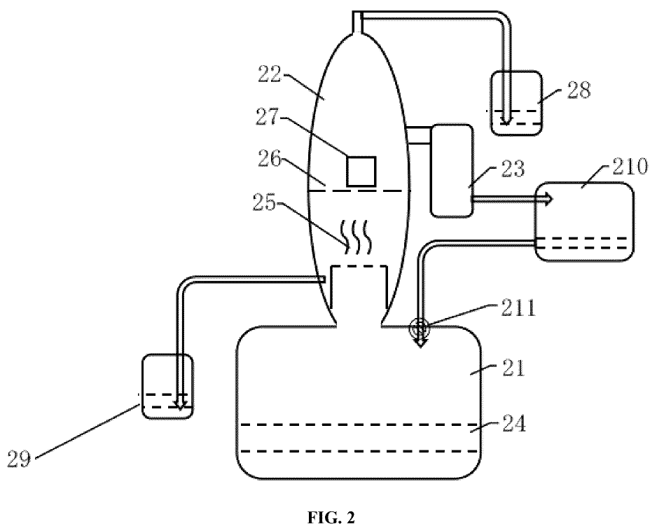

A specific implementation process of this example was carried out by using a distiller as shown in FIG. 2. In FIG. 2, the distiller includes a distillation still 21 (a high-temperature thermal control substance generating device) is equipped with a rectification tower 22 (treatment system) and a cooling tower (the pressure is changed to form one-way drive due to the volume change after the phase transition of the gaseous substance). The organic Cabernet Sauvignon grapes were cleaned, crushed, and fermented after the impurities are removed. After the fermentation broth was filtered and distilled, the primary distillate 24 with the alcohol content of 26%-29% (ABV) was added into the distillation still 21. After the distillation still 21 was heated (being heated to the phase transition temperature of the gaseous high-temperature thermal control substance, i.e., 80° C.) at atmospheric pressure, the primary distillate 24 was heated to form a gaseous ethanol-water non-azeotropic mixture 25 (the high-temperature thermal control substance, containing a small amount of impurities), and in the rectification tower 22, the gaseous ethanol-water non-azeotropic mixture 25 was in surface contact with the Xanthoceras sorbifolium flower dried at ambient temperature 27 (the low-temperature-treated substance, Xanthoceras sorbifolium flower dried in the open air) that was placed on a tower plate 26 and fed in a batch feeding mode, and cycling, mass transfer, and heat transfer were conducted. In the unidirectional cycling treatment process, the gaseous ethanol-water non-azeotropic mixture was in direct surface contact with the Xanthoceras sorbifolium flower dried at ambient temperature for mass transfer and heat transfer, part of phase transition latent heat released by the gaseous ethanol-water non-azeotropic mixture enabled the low-phase-transition-temperature component in the volatile components and the high-phase-transition-temperature component in the Xanthoceras sorbifolium flower dried at ambient temperature to be separated and collected by a low-boiling-point fraction collecting device 28 and a high-boiling-point fraction collecting device 29, respectively. Meanwhile, the surface microorganisms of the Xanthoceras sorbifolium flower dried at ambient temperature were inactivated by the gaseous ethanol-water non-azeotropic mixture. Harmful low-boiling-point fractions (i.e., low-phase-transition-temperature components) such as methanol and acetaldehyde in the volatile substances contained in the primary distillate 24 and the Xanthoceras sorbifolium flower dried at ambient temperature 27 were gathered at the top of the rectification tower 22, and collected in the low-boiling-point fraction collecting device 28 to be removed. High-boiling-point fractions (i.e., high-phase-transition-temperature components) such as higher alcohols that may cause sensory discomfort were gathered at the bottom of the rectification tower 22, and collected in the high-boiling-point fraction collecting device 29 to be removed. The original aromatic components such as alcohols, acids, and esters in the Xanthoceras sorbifolium flower dried at ambient temperature were separated and collected with the gaseous ethanol-water non-azeotropic mixture in a liquor collecting device 210, then cyclically refluxed to the distillation still 21, and entered the next substance cycle of the high-temperature thermal control substance. The gaseous ethanol-water non-azeotropic mixture 25 formed the substance cycle of the high-temperature thermal control substance among “the rectification tower 22, the cooling tower 23, the liquor collecting device 210, the valve 211, and the distillation still 1” in the treatment process. After several cycles, the original aromatic components in the Xanthoceras sorbifolium flower dried at ambient temperature were enriched in the gaseous ethanol-water non-azeotropic mixture 25, and cooled and collected in the liquor collecting device 210 to acquire Xanthoceras sorbifolium flower-flavoured brandy with a unique flavor. During implementation, the temperature of the rectification tower 22 was kept constant and below 85° C. by controlling the reflux speed of the primary distillate with the alcohol content of 26%-29% (ABV).

### Example 2 Inactivating Surface Microorganisms of Wild and Freshly-Picked Datong Daylily

This example was based on the constant-temperature dual-cycle substance separation method, and adopted the device of Example 1. Different from Example 1, the Xanthoceras sorbifolium flower dried at ambient temperature was replaced with wild and freshly-picked Datong daylily as the low-temperature-treated substance, which was used to test the effect of the constant-temperature dual-cycle substance separation method on treating surface microorganisms of fresh food. During implementation, the wild and freshly-picked Datong daylily was placed on the tower plate of the rectification tower and treated by the constant-temperature dual-cycle substance separation method. The experiment shows that the surface microorganisms of the wild and freshly-picked Datong daylily were inactivated and showed regularity. In addition, similar to the treatment result of Example 1, harmful low-boiling-point components such as methanol and acetaldehyde and high-boiling-point components such as higher alcohols that may cause sensory discomfort in the primary distillate and in the wild and freshly-picked Datong daylily were separated and removed, the original aromatic components such as alcohols, acids, and esters in the wild and freshly-picked Datong daylily were enriched to acquire Datong daylily original brandy with a unique flavor.

In the specific implementation of this example, the local wild Datong daylily freshly-picked from Datong was placed on the tower plate of the rectification tower and treated by the constant-temperature dual-cycle substance separation method. The experimental treatment time is as follows: when the ethanol-water non-azeotropic mixture in the treatment device reaches a stable cycle, a power supply was suspended, and after a sample was added into the treatment device, the power supply was turned on to start timing until the power supply of the treatment device was suspended again, the treatment time was 3 min, 6 min, 9 min and 12 min, respectively.

Under atmospheric pressure, the gaseous ethanol-water non-azeotropic mixture once containing 95.6% ethanol and 4.4% water, (generally, the gaseous ethanol-water mixture has a lower azeotropic point than a boiling point of a single component, a boiling point of ethanol is 78.3° C., a boiling point of water is 100° C.) results in that the gaseous ethanol-water mixture boils at 78.13 V, which is lower than the boiling point of ethanol or water, and much lower than 100° C.; thus the use of the gaseous ethanol-water non-azeotropic mixture for food treatment could greatly reduce the loss of nutrients in food raw materials, compared with high-temperature water steam.

### Comparative Example 1

This comparative example was performed according to procedures as described in Example 1, except that: the constant-temperature dual-cycle substance separation method was not adopted, and the gaseous ethanol-water non-azeotropic mixture formed by heating the primary distillate was directly subjected to surface contact, cycling, mass transfer, and heat transfer with the Xanthoceras sorbifolium flower dried at the ambient temperature that was placed on the tower plate in the rectification tower to obtain Xanthoceras sorbifolium flower-flavoured brandy.

In Comparative Example 1, transient extraction with the gaseous ethanol-water non-azeotropic mixture could not concentrate an aromatic substance, and meanwhile, a large amount of gaseous ethanol-water non-azeotropic mixture was consumed, resulting in failure to achieve a high level of restoration of processed food. If the ethanol-water non-azeotropic mixture alone was used for extraction, there was a risk of introducing allergic substances.

However, in Example 1, according to the constant-temperature and dual-cycle substance separation method provided by the present disclosure, the original aromatic components of the Xanthoceras sorbifolium flower dried at the ambient temperature was enriched in the gaseous ethanol-water non-azeotropic mixture 5 through multiple cycles process of the substance cycle of the high-temperature thermal control substance and the substance cycle of the low-temperature-treated substance, so as to be thoroughly separated from other impurity substances. The obtained Xanthoceras sorbifolium flower-flavoured brandy has more intense flavor than that in Comparative Example 1, and the “fully-enclosed, continuous, stable and controllable” microenvironment of the treatment system was achieved to reduce the consumption of the gaseous ethanol-water non-azeotropic mixture.

### Comparative Example 2

This Comparative Example was performed according to the procedures as described in Example 1, except that: the constant-temperature dual-cycle substance separation method was not adopted, the gaseous ethanol-water non-azeotropic mixture formed by heating the primary distillate was directly used to inactivate the surface microorganisms on the wild and freshly-picked Datong daylily placed on the tower plate in the rectification tower.

In Comparative example 2, when the gaseous ethanol-water non-azeotropic mixture was in contact with the low-temperature-treated substance, only “conduction from high temperature to low temperature” was achieved, but low-temperature dissipation was not achieved. If the low temperature of the microenvironment could not dissipate or dissipates too slowly, with the continuous addition of the low-temperature-treated substance, the temperature of the microenvironment would gradually decrease, and the inactivation effect thus decreases gradually, resulting in that some microorganisms survive. Meanwhile, the high-temperature thermal control substance may also undergo phase transition in a larger amount, making it impossible to acquire a continuous and stable microenvironment; and microenvironmental imbalance would lead to the thorough failure of microbial inactivation.

However, in Comparative Example 2, according to the constant-temperature dual-cycle substance separation method provided by the present disclosure, through the substance cycle of the high-temperature thermal control substance, the high-temperature thermal control substance took away the low temperature when exchanging heat with the low-temperature-treated substance, which realizes the low-temperature dissipation of the treatment system, thereby achieving a “fully-enclosed, continuous, stable and controllable” microenvironment of the treatment system to thoroughly inactivate the microorganisms.

### Performance Test

(1) Xanthoceras sorbifolium flower-flavoured brandy obtained by the preparation method in Example 1 was aged with oak chips as a sample group (JA), while a sample group treated in parallel without adding any Xanthoceras sorbifolium flower dried at ambient temperature was used as a blank control group (JH). The above two groups of samples were tested by gas chromatography-mass spectrometry (GC-MS) and analyzed by metabolomics methods (commissioned by Shanghai ProfLeader Biotech Co., Ltd., China). A VIP value of the first principal component of the OPLS-DA model (threshold>1) combined with a p value of the unidimensional test (threshold<0.05) was used to find differentially expressed metabolites. The results are shown in Table 1. A qualitative method of the differentially expressed metabolites used a search of a self-built standard substance database.

TABLE 1
Differential metabolites in JA group and JH group

| Metabolites | VIP | p-value | Log₂FC(JA/JH) | HMDB | KEGG |
|---|---|---|---|---|---|
| 2-Hydroxyglutaric acid | 1.13 | 3.55E−02 | 0.31 | HMDB0000694 | — |
| 3,4-dihydroxybutyric acid | 1.23 | 2.08E−03 | 0.91 | HMDB0000337 | — |
| 4-Hydroxypyridine | 1.14 | 3.36E−02 | 0.44 | — | — |
| Benzoic acid | 1.13 | 1.95E−02 | 0.80 | HMDB0001870 | C00180 |
| Cellobiose | 1.23 | 1.82E−03 | −1.05 | HMDB0000055 | C06422 |
| Decanoic acid | 1.21 | 4.84E−03 | −0.94 | HMDB0000511 | C01571 |
| Ethyl laurate | 1.22 | 4.52E−03 | −1.38 | HMDB0033788 | — |
| Ethyl linoleate | 1.23 | 1.87E−03 | −1.43 | — | — |
| Ethyl octadecanoate | 1.11 | 3.74E−02 | −0.36 | HMDB0034156 | — |
| Ethyl palmitate | 1.24 | 1.05E−03 | −1.28 | HMDB0029811 | — |
| Ethyl tartrate | 1.17 | 1.29E−02 | −0.39 | HMDB0033584 | — |
| Ethyl vanillin | 1.17 | 1.89E−02 | 0.56 | HMDB0029665 | — |
| Fructose | 1.11 | 2.94E−02 | −1.09 | HMDB0000660 | C02336 |
| Fumaric acid | 1.11 | 4.73E−02 | 0.42 | HMDB0000134 | C00122 |
| Glucose | 1.10 | 2.52E−02 | −0.81 | HMDB0000122 | C00031 |
| Glycerol | 1.14 | 3.54E−02 | 0.27 | HMDB0000131 | C00116 |
| Hexadecanoic acid | 1.17 | 1.89E−02 | −0.60 | HMDB0000220 | C00249 |
| Isoferulic acid | 1.16 | 1.95E−02 | 0.49 | HMDB0000955 | C10470 |
| Malic acid | 1.17 | 1.84E−02 | 0.65 | HMDB0000744 | C00711 |
| Malonic acid | 1.19 | 9.04E−03 | 1.09 | HMDB0000691 | C00383 |
| Maltose | 1.24 | 5.00E−04 | −4.18 | HMDB0000163 | C00208 |
| Mannobiose | 1.18 | 1.27E−02 | −1.14 | HMDB0029933 | — |
| Myo-Inositol | 1.13 | 3.33E−02 | 0.44 | HMDB0000211 | C00137 |
| Octanoic acid | 1.18 | 1.62E−02 | −0.73 | — | — |
| Oleic acid | 1.12 | 4.18E−02 | 0.38 | HMDB0000207 | C00712 |
| Pyroglutamic acid | 1.12 | 3.10E−02 | 0.60 | HMDB0000267 | C01879 |
| Tetradecanoic acid | 1.09 | 4.78E−02 | 0.45 | HMDB0000806 | C06424 |
| Threose | 1.22 | 3.44E−03 | 0.88 | HMDB0002649 | C01796 |
| Trehalose | 1.13 | 3.74E−02 | −1.96 | HMDB0000975 | C01083 |
| Vanillic acid | 1.17 | 1.90E−02 | 0.52 | HMDB0000484 | C06672 |
| β-Gentiobiose | 1.13 | 3.59E−02 | −0.95 | — | — |

NOTE: VIP variable importance in the projection, was obtained from the OPLS-DA model; Pvalue was calculated by Student's t-test; Log₂FC, fold change, was calculated as a binary logarithm of the average peak area (normalized) ratio between Group JA versus Group JH, where a positive value meant that the average mass response of the metabolite in Group JA was greater than that in Group JH, while a negative value meant that the average mass response of the metabolite in Group JA was less than that in Group JH.

As shown in Table 1, a total of 31 differential metabolites were screened and identified between the Group JA and the Group JH, of which 15 substances decreased and 16 substances increased. It is thus clear that chemical components harmful to human body are not detected in the Xanthoceras sorbifolium flower-flavoured brandy obtained by the constant-temperature dual-cycle substance separation method provided by the present disclosure, and the original aromatic components of Xanthoceras sorbifolium flower dried at the ambient temperature are enriched to form the Xanthoceras sorbifolium flower-flavoured brandy with a unique flavor.

(2) A sample in Example 2 was packaged and sent for test, and was tested by Shandong Baier Testing Co., Ltd., China according to GB4789.2-2022 (Food Microbiological Analysis: Aerobic plate count) and GB4789.15-2016 (Food Microbiological Analysis: Mold and Yeast count), with test results shown in Table 2. The results show that when the treatment time is 6 min, 9 min and 12 min, the aerobic plate count and the mold and yeast counts are each less than 10 CFU/g, indicating that the bacteria, the molds and the yeasts are “undetected” according to the above Chinese national determination standard. When the treatment time is 3 min, the test result of the aerobic plate count is 10 CFU/g, that is to say, the total bacteria colony is detected as 1 CFU/g, the test results of the mold and yeast counts are both less than 10 CFU/g, that is to say, the molds and the yeasts are “undetected”. In comparison, in the blank control group without the treatment by the constant-temperature dual-cycle substance separation method, the aerobic plate count and the mold and yeast counts in the sample are 3.1×10⁴, 1.2×10² and 1.7×10³ CFU/g, respectively. The analysis shows that the inactivation situation of the surface microorganisms of the wild and freshly-picked Datong daylily treated by the constant-temperature dual-cycle substance separation method provided by the present disclosure changes regularly, the surface microorganisms of the wild and freshly-picked Datong daylily could be inactivated by treating for 6 min, and by treating 3 min, the surface microorganisms such as bacteria of the wild and freshly-picked Datong daylily could not be completely inactivated, but the microorganisms such as molds and yeasts could be completely inactivated.

TABLE 2
Test results of inactivation situations of surface organisms
of wild and freshly-picked Datong daylily by the constant-
temperature dual-cycle substance separation method

| Test results | | Treatment time (min) | | | |
|---|---|---|---|---|---|
| (CFU/g) | Blank control | 3 | 6 | 9 | 12 |
| Total bacteria colony | 3.1 × 10⁴ | 10 | <10 | <10 | <10 |
| (GB4789.2-2022) | | | | | |
| Mold (First method of | 1.2 × 10² | <10 | <10 | <10 | <10 |
| GB4789.15-2016) | | | | | |
| Yeast (First method of | 1.7 × 10³ | <10 | <10 | <10 | <10 |
| GB4789.15-2016) | | | | | |

Noted: when the sample dilutability is “1:10”, “1:100”, “1:1000”, and the tested aerobic plate count (CFU/g) is “0, 0”, the result is expressed as “less than 10”.

Although the above embodiments have described the present disclosure in detail, they are only a part than all of the embodiments of the present disclosure. Those skilled in the art could also obtain other embodiments according to the embodiments without creative labor, and these embodiments all fall within the scope of the present disclosure.

What is claimed is:

1. A constant-temperature dual-cycle substance separation method, comprising:
carrying out two cycles in a fully-closed treatment system, wherein the two cycles comprise a substance cycle of a thermal control substance, and a substance cycle of a substance to be treated, the substance to be treated comprises a first-phase-transition-temperature component and a second-phase-transition-temperature component, and the thermal control substance has a phase transition temperature between a phase transition temperature of the first-phase-transition-temperature component and a phase transition temperature of the second-phase-transition-temperature component; wherein
the thermal control substance has a temperature higher than a temperature of the substance to be treated,
the first-phase-transition-temperature component has the phase transition temperature lower than the phase transition temperature of the second-phase-transition-temperature component,
the substance cycle of the substance to be treated is performed by:
heating the thermal control substance and then bringing a heated thermal control substance in contact with the substance to be treated for heat exchange, such that phase transition latent heat of the thermal control substance during the heat exchange enables the first-phase-transition-temperature component in the substance to be treated to undergo first-order phase transition and then separation from the second-phase-transition-temperature component, and taking out the first-phase-transition-temperature component after the separation, and
taking out the second-phase-transition-temperature component after the separation, or introducing the second-phase-transition-temperature component after the separation into a next cycle process as the substance to be treated; and
the substance cycle of the thermal control substance is performed by:
after heat exchange with the substance to be treated, reheating the thermal control substance by reflux, and then introducing a reheated thermal control substance into a next cycle process.

2. The constant-temperature dual-cycle substance separation method as claimed in claim 1, wherein each of the thermal control substance, the first-phase-transition-temperature component, and the second-phase-transition-temperature component is a substance with a first-order phase transition property.

3. The constant-temperature dual-cycle substance separation method as claimed in claim 1, wherein the substance to be treated is fed in a feeding mode selected from the group consisting of batch feeding and unidirectional continuous cyclic feeding.

4. The constant-temperature dual-cycle substance separation method as claimed in claim 1, wherein the thermal control substance is one selected from the group consisting of a gaseous substance and a liquid substance, and the gaseous substance is one selected from the group consisting of a gaseous azeotropic mixture and a gaseous non-azeotropic mixture.

5. The constant-temperature dual-cycle substance separation method as claimed in claim 1, wherein the substance to be treated exhibits fluidity; or
once not exhibiting the fluidity, the substance to be treated is pulverized to achieve the fluidity.

6. The constant-temperature dual-cycle substance separation method as claimed in claim 2, wherein under a condition that the substance with the first-order phase transition property in the fully-closed treatment system is gaseous, a phase transition temperature of the substance with the first-order phase transition property is changed by changing a pressure of the fully-closed treatment system.

7. The constant-temperature dual-cycle substance separation method as claimed in claim 1, wherein under a condition that a pressure of the fully-closed treatment system is a constant pressure, a temperature of the fully-closed treatment system is controlled by controlling at least one of a phase transition temperature property and a reflux speed of the thermal control substance.

8. The constant-temperature dual-cycle substance separation method as claimed in claim 4, wherein under a condition that the thermal control substance is the gaseous non-azeotropic mixture, a temperature of the fully-closed treatment system is controlled by controlling a proportion of each component in the thermal control substance.

9. A method for preparing Xanthoceras sorbifolium flower-flavoured brandy, comprising:
performing the steps of the constant-temperature dual-cycle substance separation method as claimed in claim 1,
wherein the thermal control substance is a gaseous ethanol-water non-azeotropic mixture at a temperature of 80° C. to 85° C., and the substance to be treated is Xanthoceras sorbifolium flower dried at ambient temperature.

10. A method for inactivating surface microorganisms of a wild and freshly-picked Datong daylily, comprising:
performing the steps of the constant-temperature dual-cycle substance separation method as claimed in claim 1,
wherein the thermal control substance is a gaseous ethanol-water non-azeotropic mixture at a temperature of 80° C. to 85° C., and the substance to be treated is the wild and freshly-picked Datong daylily.

11. The constant-temperature dual-cycle substance separation method as claimed in claim 2, wherein the thermal control substance is one selected from the group consisting of a gaseous substance and a liquid substance, and the gaseous substance is one selected from the group consisting of a gaseous azeotropic mixture and a gaseous non-azeotropic mixture.

12. The constant-temperature dual-cycle substance separation method as claimed in claim 2, wherein the substance to be treated exhibits fluidity; or
once not exhibiting the fluidity, the substance to be treated is pulverized to achieve the fluidity.
